(19) United States
(12) Patent Application Publication
(10) Pub. No.: US 2004/0176371 A1
(43) Pub. Date: Sep. 9, 2004

(54) Novel pyrazolopyrimidinethione derivatives, preparation methods thereof and their use as therapeutics for erectile dysfunction

(76) Inventors: Joong-Hyop Kim, Seoul (KR); Youseung Kim, Seoul (KR); Kyung Il Choi, Seoul (KR); Dong Hyun Kim, Seoul (KR); Gilsoo Nam, Seoul (KR); Jae Hong Seo, Seoul (KR)

Correspondence Address: Roberts Abokhair & Mardula, Suite 1000, 11800 Sunrise Valley Drive, Reston, VA, 20191-5302, US

(21) Appl. No.: 10/480,191

(22) PCT Filed: Jun. 14, 2002
(86) PCT No.: PCT/KR02/01126

(30) Foreign Application Priority Data
Jun. 14, 2001 (KR) 2001-0033382

(51) Int. Cl. A61K 31/5377; A61K 31/519; C07D 487/02

(52) U.S. Cl. 514/234.5; 514/252.16; 544/114; 544/262

(57) ABSTRACT

Novel pyrazolopyrimidinethione compounds of formula 1: wherein R₁ and R₂ are independently each hydrogen atom, a C₁-C₆ alkyl group, or a C₃-C₆ cycloalkyl group, R₃ is a C₁-C₆ alkyl group. C₃-C₆ cycloalkyl group or C₃-C₆ alkenyl group which is substituted or unsubstituted, X is O or NR₄, and R₄ is hydrogen atom, or a C₁-C₆ alkyl group, a C₃-C₆ cycloalkyl group or a C₃-C₆ alkenyl group which is unsubstituted or substituted with OH or an alkoxy group, pharmacologically acceptable salts or hydrates thereof, and preparation methods thereof are disclosed. Pharmaceutical compositions comprising the compounds are effectively used for the treatment of erectile dysfunction.

### TECHNICAL FIELD

The present invention relates to compounds of formula 1:

<Formula 1>

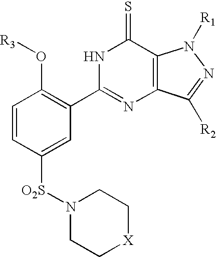

wherein

R₁ and R₂ are independently each hydrogen atom, a C₁-C₆ alkyl group, or a C₃-C₆ cycloalkyl group,

R₃ is a C₁-C₆ alkyl group, C₃-C₆ cycloalkyl group or C₃-C₆ alkenyl group which is substituted or unsubstituted,

X is O or NR₄, and

R₄ is hydrogen atom, or a C₁-C₆ alkyl group, a C₃-C₆ cycloalkyl group or a C₃-C₆ alkenyl group which is unsubstituted or substituted with OH or an alkoxy group,

pharmacologically acceptable salts or hydrates thereof as phosphodiesterase V inhibitors effective for treating erectile dysfunction, preparation methods thereof, and pharmaceutical compositions comprising the compounds for treating erectile dysfunction.

These compounds can exist in tautomeric equilibrium depicted in the following Scheme 1.

[Scheme 1]

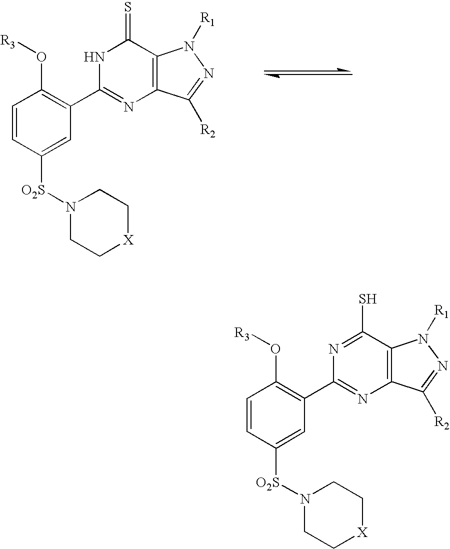

### BACKGROUND ART

Erectile dysfunction is a disease defined as the inability to achieve or maintain an erection sufficiently rigid for satisfactory sexual intercourse. A number of causes of erectile dysfunction are known, including organic factors, psycogenic factors and combinations thereof. Methods for the treatment of erectile dysfunction include use of vacuum-constriction devices, injection into the penial corpora cavernosum or intraurethral administration of a vasoactive agent such as alprostadil, implantation of penile prostheses, arterial or intravenous surgery, etc. In addition to these methods, some drugs such as yohimbin have been used for treating erectile dysfunction. However, these drugs are inconvenient in terms of their administration and their effects are unsatisfactory.

Sildenafil, a newly developed drug for the treatment of erectile dysfunction, exhibits new possibility for treating erectile dysfunction with an inhibitor of phosphodiesterase V, present in penial corpora cavernosum Pyrazolopyrimidine, the basic structural unit of sildenafil, and its inhibitory effects against phosphodiesterase V, are described in WO 96/28,448, EP 636,626, WO 93/06104, WO 93/7149, WO 94/28902 and WO 98/49166.

However, despite sildenafil's highly curative effects on erectile dysfunction, it also accompanies side effects such as acute myocardial infarction in the case of a person suffering from myocardial infarction, stroke, heart failure, arrhythmias, hypotension or hypertension. Accordingly, use of sildenafil must be cautiously made. Such undesirable side effects result from existence of 10 or more isozymes of phosphodiesterase. In particular, non-selectivity of sildenafil on phosphodiesterase VI present in eyes, phosphodiesterase m present in heart, etc., is closely associated with its side effects.

### DISCLOSURE OF THE INVENTION

Therefore, the present invention has been made in view of the above-mentioned problems, and it is an object of the present invention to provide pyrazolopyrimidinethione compounds as phosphodiesterase V inhibitors effective for the treatment of erectile dysfunction with few side effects, preparation methods thereof, and pharmaceutical compositions comprising the compounds for treating erectile dysfunction.

The present invention relates to compounds of formula 1:

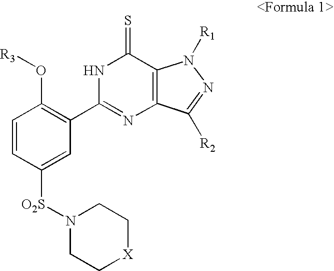

wherein

R₁ and R₂ are independently each hydrogen atom, a C₁-C₆ alkyl group or a C₃-C₆ cycloalkyl group,

R₃ is a C₁-C₆ alkyl group, a C₃-C₆ cycloalkyl group or a C₃-C₆ alkenyl group which is substituted or unsubstituted,

X is O or NR₄, and

R₄ is hydrogen atom, or a C₁-C₆ alkyl group, a C₃-C₆ cycloalkyl group or a C₃-C₆ alkenyl group which is unsubstituted or substituted with OH or an alkoxy group,

pharmacologically acceptable salts or hydrates thereof as phosphodiesterase V inhibitors effective for treating erectile dysfunction, preparation methods thereof, and pharmaceutical compositions comprising the compounds for treating erectile dysfunction.

Particularly preferred compounds of formula 1 are as follows:

1) 5-[2-methoxy-5-(4-methylpiperazine-1-sulfonyl)phenyl]-1-methyl-3-propyl-1,6 dihydropyrazolo[4,3-d]pyrimidine-7-thione (compound of Example 1),

2) 5-[2-methoxy-5-(4-ethylpiperazine-1-sulfonyl)phenyl]-1-methyl-3-propyl-1,6-dihydropyrazolo[4,3-d]pyrimidine-7-thione (compound of Example 2),

3) 5-{5-[4-(2-hydroxyethyl)piperazine-1-sulfonyl]-2-methoxyphenyl}-1-methyl-3-propyl-1,6-dihydropyrazolo[4,3-d]pyrimidine-7-thione (compound of Example 3),

4) 5-[2-methoxy-5-(morpholine-4-sulfonyl)phenyl]-1-methyl-3-propyl-1,6-dihydropyrazolo[4,3-d]pyrimidine-7-thione (compound of Example 4),

5) 5-[2-ethoxy-5-(4-methylpiperazine-1-sulfonyl)phenyl]-1-methyl-3-propyl 1,6-dihydropyrazolo[4,3-d]pyrimidine-7-thione (compound of Example 5),

6) 5-[2-ethoxy-5-(4-ethylpiperazine-1-sulfonyl)phenyl]-1-methyl-3-propyl-1,6-dihydropyrazolo[4,3-d]pyrimidine-7-thione (compound of Example 6),

7) 5-(2-ethoxy-5-[4-(2-hydroxyethyl)piperazine-1-sulfonyl]phenyl)-1-methyl-3-propyl-1,6-dihydropyrazolo[4,3-d]pyrimidine-7-thione (compound of Example 7),

8) 5-[2-ethoxy-5-(morpholine-4-sulfonyl)phenyl]-1-methyl-3-propyl-1,6-dihydropyrazolo[4,3-d]pyrimidine-7-thione (compound of Example 8),

9) 5-[2-ethoxy-5-(piperazine-1-sulfonyl)phenyl]-1-methyl-3-propyl-1,6-dihydropyrazolo[4,3-d]pyrimidine-7-thione (compound of Example 9),

10) 1-methyl-5-[5-(4-methylpiperazine-1 sulfonyl)-2-propoxyphenyl]-3-propyl-1,6-dihydropyrazolo[4,3-d]pyrimidine-7-thione (compound of Example 10),

11) 5-[5-(4-ethylpiperazine-1-sulfonyl)-2-propoxyphenyl]-1-methyl-3-propyl-1,6-dihydropyrazolo[4,3-d]pyrimidine-7-thione (compound of Example 11),

12) 5-{5-[4-(2-hydroxyethyl)piperazine-1-sulfonyl]-2-propoxyphenyl} 1-methyl-3-propyl-1,6-dihydropyrazolo[4,3-d]pyrimidine-7-thione (compound of Example 12),

13) 1-methyl-5-[5-(morpholine-4-sulfonyl)-2-propoxyphenyl]-3-propyl-1,6-dihydropyrazolo[4,3-d]pyrimidine-7-thione (compound of Example 13),

14) 5-[2-butoxy-5-(4-methylpiperazine-1-sulfonyl)phenyl]-1-methyl-3-propyl-1,6-dihydropyrazolo[4,3-d]pyrimidine-7-thione (compound of Example 14),

15) 5-[2-butoxy-5-(4-ethylpiperazine-1-sulfonyl)phenyl]-1-methyl-3-propyl-1,6-dihydropyrazolo[4,3-d]pyrimidine-7-thione (compound of Example 15),

16) 5-(2-butoxy-5-[4-(2-hydroxyethyl)piperazine-1-sulfonyl]phenyl)-1-methyl-3-propyl-1,6-dihydropyrazolo[4,3-d]pyrimidine-7-thione (compound of Example 16),

17) 5-[2-butoxy-5-(morpholine-4-sulfonyl)phenyl]-1-methyl-3-propyl-1,6-dihydropyrazolo[4,3-d]pyrimidine-7-thione (compound of Example 17),

18) 5-[2-isobutoxy-5-(4-methylpiperazine-1-sulfonyl)phenyl]-1-methyl-3-propyl-1,6-dihydropyrazolo[4,3-d]pyrimidine-7-thione (compound of Example 18),

19) 5-[2-isobutoxy-5-(4-ethylpiperazine-1-sulfonyl)phenyl]-1-methyl-3-propyl-1,6-dihydropyrazolo[4,3-d]pyrimidine-7-thione (compound of Example 19),

20) 5-(5-[4-(2-hydroxyethyl)piperazine-1-sulfonyl]-2-isobutoxyphenyl)-1-methyl-3-propyl-1,6-dihydropyrazolo[4,3-d]pyrimidine-7-thione (compound of Example 20),

21) 5-[2-isobutoxy-5-(morpholine-4-sulfonyl)phenyl]-1-methyl-3-propyl-1,6-dihydropyrazolo[4,3-d]pyrimidine-7-thione (compound of Example 21),

22) 1-methyl-5-[2-(3-methylbutoxy)-5-(4-methylpiperazine-1-sulfonyl)phenyl]-3-propyl-1,6-dihydropyrazolo[4,3-d]pyrimidine-7-thione (compound of Example 22),

23) 1-methyl-5-[2-(3-methylbutoxy)-5-(4-ethylpiperazine-1-sulfonyl)phenyl]-3-propyl-1,6-dihydropyrazolo[4,3-d]pyrimidine-7-thione (compound of Example 23),

24) 5-[5-[4-(2-hydroxyethyl)piperazine-1-sulfonyl]-2-(3-methylbutoxy)phenyl]-1-methyl-3-propyl-1,6-dihydropyrazolo[4,3-d]pyrimidine-7-thione (compound of Example 24), and

25) 1-methyl-5-[2-(3-methylbutoxy)-5-(morpholine-4-sulfonyl)phenyl]-3-propyl-1,6-dihydropyrazolo[4,3-d]pyrimidine-7-thione (compound of Example 25).

The compounds of formula 1 according to the present invention may also be pharmacologically acceptable salt forms. Examples of salts usable herein include acid addition salts and pharmacologically acceptable metal salts. The acid addition salts can be formed by suitable inorganic acids, e.g., hydrochloric acid, hydrobromic acid, sulfuric acid or phosphoric acid, or organic acid, e.g., organic carboxylic acid or organic sulfonic acid. The metal salts include alkali metal salts, preferably sodium salts or potassium salts.

The present invention also provides preparation methods of the compounds of formula 1 through the following Scheme 2 or 3.

[Scheme 2]

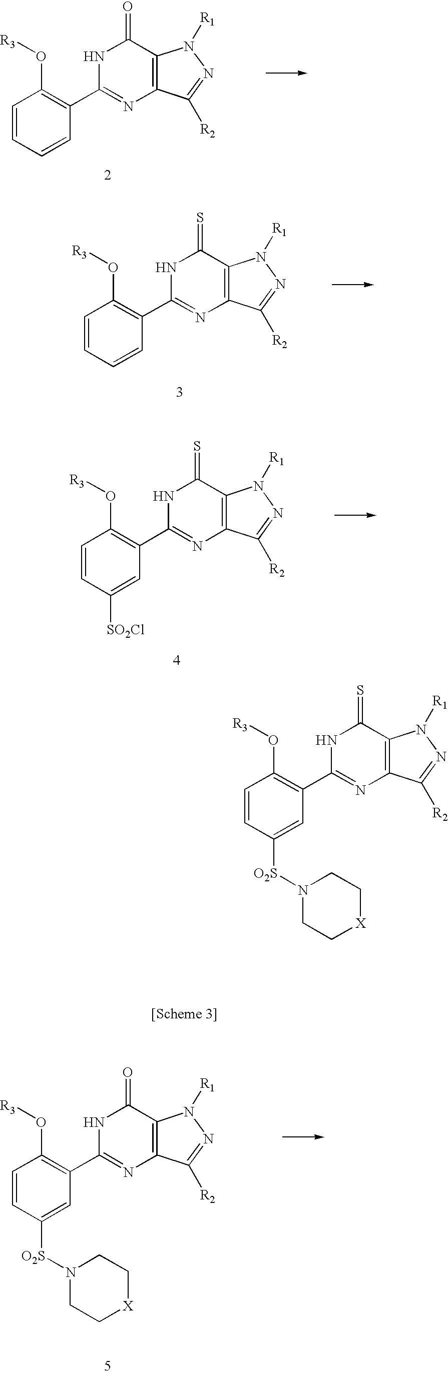

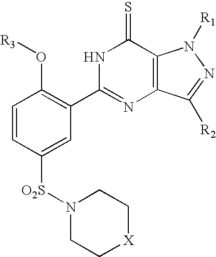

wherein

R₁, R₂, R₃ and X are as defined in formula 1.

The compound 2 can be synthesized according to the method described in WO 98/49166, and various known methods for converting pyrazolopyrimidinone into pyrazolopyrimidinethione (compound 3) can be used herein. Among these, thionation is carried out by reacting phosphorus pentasulfide, or 2,4-bis(4 methoxyphenyl)-1,3-dithia-2,4-diphosphetane-2,4-disulfide (Lawesson's reagent) or derivatives thereof in a solvent such as tetrahydrofuran, dioxane, pyridine, benzene, xylene or toluene at room temperature or refluxing temperature. Preferably, the reaction is carried out by reacting phosphorus pentasulfide in toluene at room temperature or refluxing temperature.

Chlorosulfonation of the compound 3 is carried out by stirring 5 to 20 equivalents of chlorosulfonic acid and 2 to 10 equivalents of thionyl chloride at 0° C. or at room temperature. The chlorosulfonated compound is reacted with the corresponding secondary amine in an appropriate solvent to prepare the compound of formula 1. At this time, 2 to 5 equivalents of secondary amine can be used alone, or a mixture of 1 equivalent of secondary amine and 1 to 5 equivalents of tertiary amine can be used. Examples of secondary amines usable herein include piperazine, morpholine and piperazine derivatives, and examples of tertiary amines include triethylamine and pyridine. Examples of solvents include alkanol, tetrahydrofuran, water, acetonitrile, pyridine, dimethylformamide and N,N-dimethylacetamide. 2 to 5 equivalents of secondary amine in ethanol at room temperature are preferred.

As depicted in Scheme 3, the compounds of formula 1 were also prepared from the compound 5 via the first step (thionation) of Scheme 2.

The present invention also relates to pharmaceutical compositions comprising the compounds of formula 1 as effective ingredients for treating erectile dysfunction. The pharmaceutical compositions according to the present invention may be made up in a solid form or in liquid form, and may be administered orally or parenterally. In order to prepare a pharmaceutical composition for oral administration, the compounds of formula 1 are formed into tablets or coat tablets by mixing the compounds with appropriate carriers, such as aromatics, flavorings and colorings. Examples of solid carriers include starch, lactose, mannitol, methyl cellulose, talcum, silicic acid, high molecular weight fatty acids, gelatin, agar, calcium phosphate, magnesium stearate, animal and vegetable fats, and solid polymers. The formulations for oral administration, if necessary, can contain flavorings and sweeteners.

The pharmaceutical compositions according to the present invention may be prepared in the form of suspensions or liquids by adding water or olive oil to the compounds of formula 1.

The pharmaceutical compositions according to the present invention may be used in the form of injection solutions containing stabilizers, solubilizers and buffer solutions. Other additives usable in the injection solution include tartrate or borate buffer, ethanol, dimethylsulfoxide, chelating agents, viscosity controlling polymers or polyethylene derivatives of sorbitol anhydride.

The dosage for the compounds of formula 1 according to the present invention can be varied depending upon health and body weight of the patient to be treated, and the type, frequency and desired effect of a combined treatment. The effective dosage of the compounds of formula 1 is commonly in the range of 0.01 to 100 mg/kg, and preferably 0.1 to 500 mg/kg.

The compounds of formula 1 according to the present invention show superior inhibitory activities against phosphodiesterase V as well as lower inhibitory activities against phosphodiesterase isozymes associated with side effects compared to those previously reported, in particular, sildenafil. Accordingly, the compounds of formula 1 according to the present invention can be used as drugs for the treatment of erectile dysfunction with few side effects.

The present invention will now be described in more detail with reference to the following Examples. However, these examples are given by way of illustration and not of limitation.

### BEST MODE FOR CARRYING OUT THE INVENTION

### [EXAMPLE]

### (Example 1)

Preparation of 5-[2-methoxy-5-(4-methylpiperazine-1-sulfonyl)phenyl]-1-methyl-3-propyl-1,6-dihydropyrazolo[4,3-d]pyrimidine-7-thione

(Step 1) Preparation of 5-(2-methoxyphenyl)-1-methyl-3-propyl-1,6-dihydropyrazolo[4,3-d]pyrimidine-7-thione

After 5-(2-methoxyphenyl)-1-methyl-3-propyl-1,6-dihydropyrazolo[4,3-d]pyrimidin-7-one (696.4 mg, 2.34 mmol) and phosphorus pentasulfide (114.5 mg, 0.515 mmol) were suspended in toluene (23 ml), the suspension was refluxed for 1 hour. The solvent was evaporated under reduced pressure, and dichloromethane (50 ml) and 6N-aqueous sodium hydroxide solution (10 ml) were added to the remaining residue. The organic layer was separated, dried over anhydrous magnesium sulfate, filtered, and dried under reduced pressure to prepare the title compound (675.1 mg, 92.1%) as a yellow solid.

¹H-NMR (300 MHz, CDCl₃) δ 12.34 (bs, 1H), 8.38 (d, J=7.9 Hz, 1H), 7.40 (m, 1H), 7.07 (t, J=7.6 Hz, 1H), 6.98 (d, J=8.4 Hz, 1H), 4.46 (s, 31), 4.04 (s, 3H), 2.90 (t, J=7.6 Hz, 2H), 1.88 (m, 2H), 1.03 (t, J=7.4 Hz, 3H)

¹³C-NMR (75 MHz, CDCl₃) δ 171.75, 157.37, 148.01, 146-39, 134.58, 133.05, 132.42, 131.06, 122.20, 119.17, 112.09, 56.64, 39.66, 28.01, 22.66, 14.49

(Step 2) Preparation of 4-methoxy-3-(1-methyl-3-propyl-7-thioxo-6,7-dihydro-1H-pyrazolo[4,3-d]pyrimidin-5-yl)-benzenesulfonyl chloride

Chlorosulfonic acid (2.00 ml, 30.1 mmol) was added dropwise to 5-(2-methoxyphenyl)-1-methyl-3-propyl-1,6-dihydropyrazolo[4,3-d]pyrimidine-7-thione (571.3 mg, 1.817 mmol) at 0° C. and then thionyl chloride (0.20 ml, 2.7 mmol) was added thereto. After the reaction mixture was stirred at room temperature for 12 hours, the reaction mixture was slowly dropped into ice. The resulting solid was filtered and dried under reduced pressure to prepare the title compound (724.3 mg, 96.5%) as a yellow solid.

¹H-NMR (300 MHz, CDCl₃) δ 12.31 (bs, 1H), 9.16 (d, J=2.0 Hz, 1H), 8.14 (dd, J=8.9 Hz, J′=2.2 Hz, 1H), 7.27 (d, J=8.8 Hz, 1H), 4.54 (s, 3H), 4.47 (q, J=6.9 Hz, 2H), 2.98 (t, J=7.5 Hz, 2H), 1.87 (1,2H), 1.76 (t, J=6.9 Hz, 3H), 1.04 (t, J=7.3 Hz, 3H)

¹³C-NMR (75 MHz, CDCl₃) δ 172.23, 161.32, 147.19, 145.66, 138.11, 134.09, 132.77, 131.63, 131.08, 120.92, 114.01, 67.39, 39.85, 27.87, 22.68, 15.02, 14.41

(Step 3) Preparation of 5-[2-methoxy-5-(4-methylpiperazine-1-sulfonyl)phenyl]-1-methyl-3-propyl-1,6-dihydropyrazolo[4,3-d]pyrimidine-7-thione

4-methoxy-3-(1-methyl-3-propyl-7-thioxo-6,7-dihydro-1H-pyrazolo[4,3-d]pyrimidin-5-yl)-benzenesulfonyl chloride (104.6 mg, 0.2533 mmol) was suspended in ethanol (10 ml), and then 1-methylpiperazine (0.10 ml, 0.90 mmol) was added thereto. After the suspension was stirred at room temperature for 12 hours, ethylacetate (50 ml) and saturated aqueous sodium bicarbonate solution (20 ml) were added thereto. The organic layer was separated, dried over anhydrous magnesium sulfate, filtered and distilled under reduced pressure. The resulting residue was purified by silica gel column chromatography to prepare the title compound (104.3 mg, 86.4%) as a yellow solid.

¹H-NMR (300 MHz, CDCl₃) δ 12.18 (bs, 1H), 8.78 (d, J=2.4 Hz, 1H), 7.86 (dd, J=8.8 Hz, J′=2.4 Hz, 1H), 7.22 (d, J=8.8 Hz, 1H), 4.52 (s, 3H), 4.19 (s, 3H), 3.10 (bs, 4H), 2.95 (t, J=7.5 Hz, 2H), 2.50 (m, 4H), 2.27 (s, 3H), 1.85 (m, 2H), 1.02 (t, J=7.4 Hz, 3H)

¹³C-NMR (75 MHz, CDCl₃) δ 172.08, 160.31, 146.85, 146.35, 134.22, 132.64, 132.47, 131.21, 129.51, 120.47, 112.84, 57.51, 54.37, 46.30, 46.09, 39.81, 27.96, 22.55, 14.49

### (Example 2)

Preparation of 5-[2-methoxy-5-(4-ethylpiperazine-1-sulfonyl)phenyl]-1-methyl-3-propyl-1,6-dihydropyrazolo[4,3-d]pyrimidine-7-thione

The title compound was prepared from the title compound prepared in Step 2 of Example 1 and 1-ethylpiperazine, in accordance with Step 3 of Example 1. (Yield: 92.3%)

¹H-NMR (300 MHz, CDCl₃) δ 12.15 (bs, 1H), 8.80 (d, J=2.4 Hz, 1H), 7.87 (dd, J=8.8 Hz, J′=2.4 Hz, 1H), 7.22 (d, J=8.8 Hz, 1H), 4.52 (s, 3H), 4.19 (s, 3H), 3.11 (bs, 4H), 2.95 (t, J=7.5 Hz, 2H), 2.55 (m, 4H), 2.41 (q, J=7.2 Hz, 2H), 1.85 (m, 2H), 1.05-0.99 (m, 6H)

¹³C-NMR (75 MHz, CDCl₃) δ 172.10, 160.31, 146.88, 146.34, 134.25, 132.65, 132.54, 131.31, 129.43, 120.46, 112.81, 57.52, 52.27, 52.15, 46.44, 39.83, 27.97, 22.57, 14.42, 12.32

### (Example 3)

Preparation of 5-{5-[4-(2-hydroxyethyl)piperazine-1-sulfonyl]-2-methoxyphenyl}-1-methyl-3-propyl-1,6-dihydropyrazolo[4,3-d]pyrimidine-7-thione

The title compound was prepared from the title compound prepared in Step 2 of Example 1 and 1-(2-hydroxyethyl)piperazine, in accordance with Step 3 of Example 1. (Yield: 78.0%)

¹H-NMR (300 MHz, CDCl₃) δ 12.12 (bs, 1H), 8.79 (d, J=2.4 Hz, 1H), 7.87(dd, J=8.8 Hz, f=2.4 Hz, 1H), 7.23 (d, J=8.8 Hz, 1H), 4.52 (s, 3H), 4.20 (s, 3H), 3.57 (t, J=5.3 Hz, 2H), 3.10 (bs, 4H), 2.95 (t, J=7.5 Hz, 2H), 2.62 (m, 4H), 2.55 (t, J=5.3 Hz, 2H), 2.40 (bs, 1H), 1.86 (r, 2H), 1.02 (t, J=7.4 Hz, 3H)

¹³C-NMR (75 MHz, CDCl₃) δ 172.11, 160.38, 146.87, 146.37, 134.23, 132.66, 132.46, 131.26, 129.49, 120.61, 112.90, 59.34, 58.17, 57.51, 52.34, 46.50, 39.84, 27.95, 22.56, 14.43

### (Example 4)

Preparation of 5-[2-methoxy-5-(morpholine-4-sulfonyl)phenyl]-1-methyl-3-propyl-1,6-dihydropyrazolo[4,3-d]pyrimidine-7-thione

The title compound-was prepared from the title compound prepared in Step 2 of Example 1 and morpholine, in accordance with Step 3 of Example 1. (Yield: 91.4%)

¹H-NMR (300 MHz, CDCl₃) δ 12.10 (bs, 1H), 8.82 (d, J=2.4 Hz, 1H), 7.89 (dd, J=8.8 Hz, J′=2.4 Hz, 1H), 7.24 (d, J=8.8 Hz, 1H), 4.53 (s, 3H), 4.21 (s, 3H), 3.77 (1,4H), 3.07 (m, 4H), 2.95 (t, J=7.5 Hz, 2H), 1.86 (m, 2H), 1.02 (t, J=7.4 Hz, 3H)

¹³C-NMR (75 MHz, CDCl₃) δ 172.17, 160.44, 146.93, 146.31, 134.25, 132.71, 132.51, 131.35, 129.46, 120.67, 112.91, 66.49, 57.53, 46.39, 39.86, 27.97, 22.58, 14.41

### (Example 5)

Preparation of 5-[2-ethoxy-5-(4-methylpiperazine-1-sulfonyl)phenyl]-1-methyl-3-propyl-1,6-dihydropyrazolo[4,3-d]pyrimidine-7-thione

(Step 1) Preparation of 5-(2-ethoxyphenyl)-1-methyl-3-propyl-1,6-dihydropyrazolo[4,3-d]pyrimidine-7-thione

The title compound was prepared from 5-(2-ethoxyphenyl)-1-methyl-3-propyl-1,6-dihydropyrazolo[4,3-d]pyrimidin-7-one in accordance with Step 1 of Example 1. (Yield: 94.4%)

¹H-NMR (300 MHz, CDCl₃) δ 12.71 (bs, 1H), 8.47 (dd, J=7.9 Hz, J′=1.5 Hz, 1H), 7.45 (m, 1H), 7.13 (t, J=7.6 Hz, 1H), 7.03 (d, f=8.3 Hz, 1H), 4.52 (s, 3H), 4.29 (q, J=6.9 Hz, 2H), 2.94 (t, J=7.6 Hz, 2H), 1.86 (m, 2H), 1.68 (t, J=7.0 Hz, 3H), 1.03 (t, J=7.4 Hz, 3H)

¹³C-NMR (75 MHz, CDCl₃) δ 172.02, 157.02, 148.31, 146.53, 134.78, 133.17, 132.60, 131.09, 122.24, 119.17, 113.09, 65.86, 39.73, 28.07, 22.74, 15.28, 14.50

(Step 2) Preparation of 4-methoxy-3-(1-methyl-3-propyl-7-thioxo-6,7-dihydro-1H-pyrazolo[4,3-d]pyrimidin-5-yl)-benzenesulfonyl chloride

The title compound was prepared from 5-(2-ethoxyphenyl)-1-methyl-3 propyl-1,6-dihydropyrazolo[4,3-d]pyrimidine-7-thione in accordance with Step 2 of Example 1. (Yield: 93.4%)

¹H-NMR (300 MHz, CDCl₃) δ 12.01 (bs, 1H), 9.09 (d, J=2.5 Hz, 1H), 8.15 (dd, J=8.9 Hz, J′=2.5 Hz, 1H), 7.31 (d, J=8.9 Hz, 1H), 4.51 (s, 3H), 4.26 (s, 3H), 2.96 (t, J=7.5 Hz, 2H), 1.86 (m, 2H), 1.04 (t, J=7.4 Hz, 3H)

¹³C-NMR (75 MHz, CDCl₃) δ 172.07, 161.81, 147.09, 145.53, 138.16, 133.95, 132.67, 131.69, 131.04, 121.17, 113.48, 57.97, 46.39, 39.87, 27.85, 22.62, 14.41

(Step 3) Preparation of 5-[2-ethoxy-5-(4-methylpiperazine-1-sulfonyl)phenyl] 1-methyl-3-propyl-1,6-dihydropyrazolo[4,3-d]pyridine-7-thione

Method A:

The title compound was prepared from 4-methoxy-3-(1-methyl-3-propyl-7-thioxo-6,7-dihydro-1H-pyrazolo[4,3-d]pyrimidin-5-yl)-benzenesulfonyl chloride and 1-methylpiperazine, in accordance with Step 3 of Example 1. (Yield: 96.0%)

Method B:

5-[2-ethoxy-5-(4-methylpiperazine-1-sulfonyl)phenyl]-1-methyl-3-propyl-1,6-dihydropyrazolo[4,3-d]pyrimidin-7-one (WO 98/49166) (0.200 g, 0.421 mmol) and phosphorus pentasulfide (0.0206 g, 0.0962 mmol) were suspended in toluene (5.0 ml). The suspension was refluxed for 2 hours. After the solvent was evaporated under reduced pressure, dichloromethane (50 ml) and 6N-aqueous sodium hydroxide solution (10 ml) was added to the resulting residue. The organic layer was separated, dried over anhydrous magnesium sulfate, filtered, and dried under reduced pressure to prepare the title compound (0.160 g, 77.7%).

¹H-NMR (300 MHz, CDCl₃) δ 12.41 (s, 1H), 8.82 (d, J=2.3 Hz, 1H), 7.82 (dd, J=8.7 Hz, f=2.3 Hz, 1H), 7.19 (d, J=8.8 Hz, 1H), 4.51 (s, 3H), 4.41 (q, J=6.9 Hz, 2H), 3.10 (bs, 4H), 2.95 (t, J=7.5 Hz, 2H), 2.50 (m, 4H), 2.27 (s, 3H), 1.86 (m, 2H), 1.72 (t, J=6.9 Hz, 3H), 1.02 (t, J=7.4 Hz, 3H)

¹³C-NMR (75 MHz, CDCl₃) δ 172.09, 159.83, 146.81, 146.43, 134.23, 132.64, 132.35, 131.03, 129.29, 120.09, 113.56, 66.81, 54.38, 46.31, 46.09, 39.74, 27.97, 22.53, 15.05, 14.41

### (Example 6)

Preparation of 5-[2-ethoxy-5-(4-ethylpiperazine-1-sulfonyl)phenyl]-1-methyl-3-propyl-1,6-dihydropyrazolo[4,3-d]pyrimidine-7-thione

The title compound was prepared from the title compound prepared in Step 2 of Example 5 and 1-ethylpiperazine, in accordance with Step 3 of Example 1. (Yield: 63.6%)

¹H-NMR (300 MHz, CDCl₃) δ 12.41 (s, 1H), 8.84 (d, J=2.3 Hz, 1H), 7.84 (dd, J=8.7 Hz, r=2.3 Hz, 1H), 7.18 (d, J=8.8 Hz, 1H), 4.52 (s, 3H), 4.40 (q, J=7.0 Hz, 2H), 3.10 (bs, 4H), 2.95 (t, J=7.5 Hz, 2H), 2.54 (m, 4H), 2.41 (q, J=7.2 Hz, 2H), 1.86 (m, 2H), 1.73 (t, J=6.9 Hz, 3H), 1.05-0.99 (m, 6H)

¹³C-NMR (75 MHz, CDCl₃) δ 172.15, 159.83, 146.88, 146.44, 134.30, 132.68, 132.45, 131.19, 129.22, 120.14, 113.51, 66.83, 52.29, 52.18, 46.48, 39.77, 27.98, 22.57, 15.08, 12.35

### (Example 7)

Preparation of 5-{2-ethoxy-5-[4-(2-hydroxyethyl)piperazine-1-sulfonyl]phenyl}-1-methyl-3-propyl-1,6-dihydropyrazolo[4,3-d]pyrimidine-7-thione

The title compound was prepared from the title compound prepared in Step 2 of Example 5 and 1-(2-hydroxyethyl)piperazine, in accordance with Step 3 of Example 1. (Yield: 56.6%)

¹H-NMR (300 MHz, CDCl₃) δ 12.41 (bs, 1H), 8.82 (d, J=2.3 Hz, 1H), 7.84(dd, J=8.7 Hz, J′=2.4 Hz, 1H), 7.20 (d, J=8.8 Hz, 1H), 4.52 (s, 3H), 4.40 (t, J=6.9 Hz, 2H), 3.57 (t, J=5.2 Hz, 2H), 3.09 (bs, 4H), 2.95 (t, f=7.5 Hz, 2H), 2.61 (m, 4H), 2.55 (t, J=5.3 Hz, 2H), 2.40 (bs, 1H), 1.86 (m, 2H), 1.73 (t, J=6.9 Hz, 3H) 1.02 (t, J=7.4 Hz, 3H)

¹³C-NMR (75 MHz, CDCl₃) δ 172.14, 159.91, 146.85, 146.47, 134.26, 132.67, 132.37, 131.11, 129.16, 120.25, 113.58, 66.82, 59.33, 58.16, 52.33, 46.51, 39.78, 27.96, 22.56, 15.08, 14.43

### (Example 8)

Preparation of 5-[2-ethoxy-5-(morpholine-4-sulfonyl)phenyl]-1-methyl-3-propyl-1,6-dihydropyrazolo[4,3-d]pyrimidine-7-thione

The title compound was prepared from the title compound prepared in Step 2 of Example 5 and morpholine, in accordance with Step 3 of Example 1. (Yield: 63.4%)

¹H-NMR (300 MHz, CDCl₃) δ 12.39 (bs, 1H), 8.83 (d, J=2.4 Hz, 1H), 7.84 (dd, J=8.7 Hz, J′=2.4 Hz, 1H), 7.21 (d, J=8.8 Hz, 1H), 4.52 (s, 3H), 4.42 (q, J=6.9 Hz, 2), 3.77 (m, 4H), 3.06 (m, 4H), 2.95 (t, J=7.5 Hz, 2H), 1.86 (n, 2H), 1.73 (t, J=6.9 Hz, 3H) 1.02 (t, J=7.4 Hz, 3H)

¹³C-NMR (75 MHz, CDCl₃) δ 172.16, 159.99, 146.86, 146.41, 134.25, 132.69, 132.38, 131.13, 129.11, 120.33, 113.64, 66.85, 66.47, 46.39, 39.78, 27.97, 22.56, 15.08, 14.41

### (Example 9)

Preparation of 5-[2-ethoxy-5-(piperazine-1-sulfonyl)phenyl]-1-methyl-3-propyl-1,6-dihydropyrazolo[4,3-d]pyrimidine-7-thione

The title compound was prepared from the title compound prepared in Step 2 of Example 1 and piperazine, in accordance with Step 3 of Example 1. (Yield: 80.5%)

¹H-NMR (300 MHz, CDCl₃) δ 8.84 (d, J=2.4 Hz, 1H), 7.85 (dd, J=8.7 Hz, J′=2.4 Hz, 1H), 7.19 (d, J=8.8 Hz, 1H), 4.53 (s, 3H), 4.41 (q, J=7.0 Hz, 2H), 3.07 (m, 4H), 2.95 (m, 6H), 1.82 (m, 2H), 1.73 (t, J=7.0 Hz, 3H) 1.01 (t, J=7.4 Hz, 3H)

¹³C-NMR (75 MHz, CDCl₃) δ 172.24, 159.82, 146.91, 146.52, 134.33, 132.74, 132.36, 131.16, 129.79, 120.33, 113.51, 66.81, 47.24, 45.70, 39.77, 27.98, 22.55, 15.07, 14.38

### (Example 10)

Preparation of 1-methyl-5-[5-(4-methylpiperazine-1-sulfonyl)-2-propoxyphenyl]-3-propyl-1,6-dihydropyrazolo[4,3-d]pyrimidine-7-thione

(Step 1) Preparation of 1-methyl-5-(2-propoxyphenyl)-3-propyl-1,6-dihydropyrazolo[4,3-d]pyrimidine-7-thione

The title compound was prepared from 1-methyl-5-(2-propoxyphenyl)-3-propyl-1,6-dihydropyrazolo[4,3-d]pyrimidin-7-one in accordance with Step 1 of Example 1. (Yield: 88.0%)

¹H-NMR (300 MHz, CDCl₃) δ 12.64 (bs, 1H), 8.46 (d, J=7.9 Hz, 1H), 7.45 (t, J=8.4 Hz, 1H), 7.13 (t, J=7.5 Hz, 1H), 7.04 (d, J=8.4 Hz, 1H), 4.52 (s, 3H), 4.18 (t, J=6.5 Hz, 2H), 2.94 (t, J=7.4 Hz, 2H), 2.6 (m, 2H), 1.85 (m, 2H), 1.18 (t, J=7.4 Hz, 3H), 1.02 (t, J=7.4 Hz, 3H)

¹³C-NMR (75 MHz, CDCl₃) δ 171.90, 157.17, 148.34, 146.52, 134.74, 133.14, 132.63, 131.14, 122.18, 119.33, 113.06, 71.80, 39.71, 28.05, 22.90, 22.70, 14.45, 11.27

(Step 2) Preparation of 3-(1-methyl-3-propyl-7-thioxo-6,7-dihydro-1H-pyrazolo[4,3-d]pyrimidin-5-yl)-4-propoxybenzenesulfonyl chloride

The title compound was prepared from 1-methyl-5-(2-propoxyphenyl)-3-propyl-1,6-dihydropyrazolo[4,3-d]pyrimidine-7-thione in accordance with Step 2 of Example 1. (Yield: 99.8%)

¹H-NMR (300 MHz, CDCl₃) δ 12.28 (bs, 1H), 9.12 (d, J=2.34 Hz, 1H), 8.13 (dd, J=2.3 Hz, 6.5 Hz, 1H) 7.2 (s, 1H), 4.53 (s, 3H) 4.35 (t, J=6.4 Hz, 2H), 2.96 (t, J=7.5 Hz, 2H), 2.15 (n, 2H), 1.85 (n, 2H), 1.22 (t, J=7.4 Hz, 3H), 1.03 (t, J=7.3 Hz, 3H)

¹³C-NMR (75 MHz, CDCl₃) δ 172.32, 161.56, 147.07, 145.92, 138.00, 133.72, 132.78, 131.72, 131.07, 120.82, 114.12, 73.25, 39.81, 27.76, 22.69, 22.63, 14.37, 11.16

(Step 3) Preparation of 5-[5-(4-methylpiperazine-1-sulfonyl)-2-propoxyphenyl]-1-methyl-3-propyl-1,6-dihydropyrazolo[4,3-d]pyrimidine-7-thione

The title compound was prepared from 3-(1-methyl-3-propyl-7-thioxo-6,7-dihydro-1H-pyrazolo[4,3-d]pyrimidin-5-yl)-4-propoxybenzenesulfonyl chloride and 1-methylpiperazine in accordance with Step 3 of Example 1. (Yield: 69.9%)

¹H-NMR (300 MHz, CDCl₃) δ 12.35 (bs, 1H), 8.84 (d, J=1.35 Hz, 1H), 7.84 (dd, J=8.7, 1.3 Hz, 1H), 7.20 (d J=7.7 Hz, 1H), 4.52 (s, 3H), 4.28 (t, 6.4 Hz, 2H), 3.09 (bs, 4H), 2.94 (t, J=7.4 Hz, 2H), 2.49 (bs 4H), 2.27 (s, 3H), 2.13 (m, 2H), 1.86 (m, 2H), 1.21 (t, J=7.4 Hz, 3H), 1.01 (t, J=7.2 Hz, 3H)

¹³C-NMR (75 MHz, CDCl₃) δ 172.21, 159.99, 146.87, 146.48, 134.26, 132.71, 132.37, 131.18, 129.42, 120.28, 113.51, 72.69, 54.42, 46.35, 46.12, 39.77, 27.98, 22.73, 22.56, 14.40, 11.18

### (Example 11)

Preparation of 5-[5-(4-ethylpiperazine-1-sulfonyl)-2-propoxyphenyl]-1-methyl-3-propyl-1,6-dihydropyrazolo[4,3-d]pyridine-7-thione

The title compound was prepared from the title compound prepared in Step 2 of Example 10 and 1-ethylpiperazine, in accordance with Step 3 of Example 1. (Yield: 67.0%)

¹H-NMR (300 MHz, CDCl₃) δ 12.35 (bs, 1H), 8.86 (d, J=2.4 Hz, 1H), 7.85 (dd, J=8.7, 2.4 Hz, 111), 7.19 (d J=8.8 Hz, 1H), 4.53 (s, 31), 4.28 (t, 6.5 Hz, 2H), 3.11 (bs, 4H), 2.94 (t, J=7.4 Hz, 2H), 2.54 (bs 4H), 2.41 (q, J=7.2 Hz, 2H), 2.13 (m, 2H), 1.86 (m, 2H), 1.21 (t, J=7.4 Hz, 3H), 1.01 (m, 6H)

¹³C-NMR (75 MHz, CDCl₃) δ 172.27, 159.99, 146.93, 146.49, 134.31, 132.76, 132.45, 131.33, 129.42, 120.35, 113.46, 72.72, 52.30, 52.20, 46.49, 39.79, 27.99, 22.75, 22.58, 14.40, 12.33, 11.18

### (Example 12)

Preparation of 5-{5-[4-(2-hydroxyethyl)piperazine-1-sulfonyl]-2-propoxyphenyl}-1-methyl-3-propyl-1,6-dihydropyrazolo[4,3-d]pyrimidine-7-thione

The title compound was prepared from the title compound prepared in Step 2 of Example 10 and 1-(2-hydroxyethyl)piperazine, in accordance with Step 3 of Example 1. (Yield: 74.0%)

¹H-NMR (300 MHz, CDCl₃) δ 12.35 (bs, 1H), 8.82 (d, J=2.2 Hz, 1H), 7.84 (dd, J=8.7, 2.2 Hz, 1H), 7.20 (d J=8.8 Hz, 1H), 4.52 (s, 3H), 4.29 (t, 6.4 Hz, 2H), 3.57 (t, J=5.1 Hz, 2H), 3.09 (bs, 4H), 2.95 (t, J=7.4 Hz, 2H), 2.61 (bs, 4H), 2.55 (t, J=5.2 Hz, 2H), 2.13 (m, 2M), 1.86 (m, 2H), 1.21 (t, J=7.3 Hz, 3H), 1.01 (t, J=7.3 Hz, 3H)

¹³C-NMR (75 MHz, CDCl₃) δ 172.19, 160.06, 146.85, 146.50, 134.23, 132.70, 132.38, 131.16, 129.16, 120.37, 113.58, 72.68, 59.32, 58.15, 52.33, 46.51, 39.79, 27.96, 22.75, 22.57, 14.42, 11.22

### (Example 13)

Preparation of 1-methyl-5-[5-(morpholine-4-sulfonyl)-2-propoxyphenyl]-3-propyl-1,6-dihydropyrazolo[4,3-d]pyrimidine-7-thione

The title compound was prepared from the title compound prepared in Step 2 of Example 10 and morpholine, in accordance with Step 3 of Example 1. (Yield: 64.3%)

¹H-NMR (300 MHz, CDCl₃) δ 12.34 (bs, 1H), 8.83 (d, J=2.4 Hz, 114), 7.85 (dd, J=8.8 Hz, f=2.4 Hz, 1H), 7.22 (d, J=8.8 Hz, 1), 4.53 (s, 3H), 4.30 (t, J=6.5 Hz, 2H), 3.77 (m, 4H), 3.06 (m, 4H), 2.95 (t, J=7.5 Hz, 2H), 1.86 (m, 2H), 1.22 (t, J=7.4 Hz, 3H) 1.02 (t, J=7.4 Hz, 3H)

¹³C-NMR (75 MHz, CDCl₃) δ 172.23, 160.16, 146.88, 146.45, 134.23, 132.72, 132.40, 131.20, 129.07, 120.45, 113.63, 72.72, 66.48, 46.40, 39.80, 27.98, 22.76, 22.58, 14.41, 11.22

### (Example 14)

Preparation of 5-[2-butoxy-5-(4-methylpiperazine-1-sulfonyl)phenyl]-1-methyl-3-propyl-1,6-dihydropyrazolo[4,3-d]pyrimidine-7-thione

(Step 1) Preparation of 5-(2-butoxyphenyl)-1-methyl-3-propyl-1,6-dihydropyrazolo[4,3-d]pyrimidine-7-thione

The title compound was prepared from 5-(2-butoxyphenyl)-1-methyl-3-propyl-1,6-dihydropyrazolo[4,3-d]pyrimidin-7-one in accordance with Step 1 of Example 1. (Yield: 92.0%)

¹H-NMR (300 MHz, CDCl₃) δ 12.68 (s, 1H), 8.50 (dd, J=7.9 Hz, J′=1.6 Hz, 1H), 7.50-7.45 (m, 1H), 7.15 (m, 1H), 7.07 (d, J=8.3 Hz, 1H), 4.53 (s, 3), 4.24 (t, J=6.3 Hz, 2H), 2.95 (t, J=7.6 Hz, 2H), 2.04 (1,2H), 1.87 (1,2H), 1.65 (m, 2H), 1.07-1.00 (m, 6H)

¹³C-NMR (75 MHz, CDCl₃) δ 172.20, 157.25, 148.39, 146.57, 134.79, 133.17, 132.70, 131.20, 122.21, 119.41, 113.08, 70.03, 39.72, 31.50, 28.07, 22.71, 20.04, 14.44, 14.19

(Step 2) Preparation of 4-butoxy-3-(1-methyl-3-propyl-7-thioxo-6,7-dihydro-1H-pyrazolo[4,3-d]pyrimidin-5-yl)benzenesulfonyl chloride

The title compound was prepared from 5-(2-butoxyphenyl)-1-methyl-3-propyl-1,6-dihydropyrazolo[4,3-d]pyrimidine-7-thione in accordance with Step 2 of Example 1. (Yield: 100.0%)

¹H-NMR (300 MHz, CDCl₃) δ 12.27 (s, 1H), 9.15 (d, J=2.4 Hz, 1H), 8.13 (dd, J=9.0 Hz, J′=2.5 Hz, 1H), 7.27 (d, J=9.0 Hz, 1H), 4.53 (s, 3H), 4.39 (t, J=6.3 Hz, 21H), 2.98 (t, J=7.5 Hz, 2H), 2.09 (m, 2H), 1.87 (1,2H), 1.67 (m, 2H), 1.09-1.02 (1,6H)

¹³C-NMR (75 MHz, CDCl₃) δ 172.30, 161.55, 147.15, 145.73, 138.03, 133.97, 132.79, 131.62, 131.06, 120.95, 114.04, 71.52, 39.82, 31.21, 27.85, 22.66, 19.96, 14.38, 14.11

(Step 3) Preparation of 5-[2-butoxy-5-(4-methylpiperazine-1-sulfonyl)phenyl]-1-methyl-3-propyl-1,6-dihydropyrazolo[4,3-d]pyrimidine-7-thione

The title compound was prepared from 4-butoxy-3-(1-methyl-3-propyl-7-thioxo-6,7-dihydro-1H-pyrazolo[4,3-d]pyrimidin-5-yl)benzenesulfonyl chloride and 1-methylpiperazine in accordance with Step 3 of Example 1. (Yield: 78.9%)

¹H-NMR (300 MHz, CDCl₃) δ 12.38 (bs, 1H), 8.83 (d, J=2.4 Hz, 1H), 7.83 (dd, J=8.7 Hz, f=2.4 Hz, 1H), 7.20 (d, J=8.8 Hz, 1H), 4.52 (s, 3H), 4.33 (t, J=6.2 Hz, 2H), 3.10 (bs, 4H), 3.00 (t, J=7.5 Hz, 2H) 2.50 (m), 4H), 2.27 (s, 3H), 2.06 (m, 2H), 1.86 (m, 2H), 1.67 (m, 2H), 1.09-0.99 (m, 6H)

¹³C-NMR (75 MHz, CDCl₃) δ 172.19, 160.03, 146.84, 146.47, 134.22, 132.70, 132.37, 131.10, 129.27, 120.19, 113.52, 70.93, 54.41, 46.34, 46.12, 39.75, 31.26, 27.98, 22.55, 19.95, 14.41, 14.13

### (Example 15)

Preparation of 5-[2-butoxy-5-(4-ethylpiperazine-1-sulfonyl)phenyl]-1-methyl-3-propyl-1,6-dihydropyrazolo[4,3-d]pyrimidine-7-thione

The title compound was prepared from the title compound prepared in Step 2 of Example 14 and 1-ethylpiperazine, in accordance with Step 3 of Example 1. (Yield: 74.4%)

¹H-NMR (300 MHz, CDCl₃) δ 12.38 (s, 1H), 8.85 (d, J=2.4 Hz, 1H), 7.85 (dd, J=8.7 Hz, f=2.4 Hz, 1H), 7.20 (d, J=8.8 Hz, 1H), 4.53 (s, 3H), 4.34 (t, J=6.3 Hz, 2H), 3.11 (bs, 4H), 2.96 (t, J=7.5 Hz, 2H), 2.55 (m, 4H), 2.42 (q, J=7.2 Hz, 2H), 2.07 (m), 2H), 1.87 (1,2H), 1.68 (m, 2H), 1.10-1.00 (m, 9H)

¹³C-NMR (75 MHz, CDCl₃) δ 172.21, 160.03, 146.86, 146.47, 134.24, 132.71, 132.44, 131.19, 129.13, 120.19, 113.49, 70.94, 52.28, 52.17, 46.48, 39.76, 31.26, 27.98, 22.56, 19.95, 14.41, 14.13, 12.34

### (Example 16)

Preparation of 5-(2-butoxy-5-[4-(2-hydroxyethyl)piperazine-1-sulfonyl]phenyl)-1-methyl-3-propyl-1,6-dihydropyrazolo[4,3-d]pyrimidine-7-thione

The title compound was prepared from the title compound prepared in Step 2 of Example 14 and 1-(2-hydroxyethyl)piperazine, in accordance with Step 3 of Example 1. (Yield: 67.8%)

¹H-NMR (300 MHz, CDCl₃) δ 12.38 (bs, 1H), 8.84 (d, J=2.4 Hz, 1H), 7.85(dd, J=8.7 Hz, J′=2.4 Hz, 1H), 7.21 (d, J=8.8 Hz, 1H), 4.53 (s, 3H), 4.34 (t, J=6.2 Hz, 2H), 3.57 (t, J=5.2 Hz, 2H), 3.10 (bs, 4H), 2.95 (t, J=7.5 Hz, 2H), 2.62 (m, 4H), 2.55 (t, J=5.3 Hz, 2H), 2.39 (bs, 1H), 1.86 (m, 2H), 1.67 (m, 2H), 1.09-0.99 (m, 6H)

¹³C-NMR (75 MHz, CDCl₃) δ 172.25, 160.11, 146.88, 146.50, 134.25, 132.74, 132.38, 131.17, 129.25, 120.37, 113.54, 70.95, 59.33, 58.16, 52.35, 39.78, 31.29, 27.97, 22.58, 19.98, 14.42, 14.14

### (Example 17)

Preparation of 5-[2-butoxy-5-(morpholine-4-sulfonyl)phenyl]-1-methyl-3-propyl-1,6-dihydropyrazolo[4,3-d]pyrimidine-7-thione

The title compound was prepared from the title compound prepared in Step 2 of Example 14 and morpholine, in accordance with Step 3 of Example 1. (Yield: 82.9%)

¹H-NMR (300 MHz, CDCl₃) δ 12.36 (bs, 1H), 8.83 (d, J=2.4 Hz, 1H), 7.84 (dd, J=8.8 Hz, J′=2.4 Hz, 1H), 7.22 (d, J=8.8 Hz, 1H), 4.52 (s, 3H), 4.34 (t, J=6.2 Hz, 2H), 3.77 (1,4H), 3.06 (m, 4H), 2.95 (t, J=7.5 Hz, 2H), 2.07 (m, 2H), 1.86 (m, 2H), 1.67 (m, 2H), 1.09-0.99 (m, 6H)

¹³C-NMR (75 MHz, CDCl₃) δ 172.23, 160.20, 146.86, 146.45, 134.20, 132.73, 132.39, 131.15, 129.03, 120.40, 113.62, 70.97, 66.47, 46.39, 39.77, 31.28, 27.97, 22.56, 19.97, 14.10, 14.14

### (Example 18)

Preparation of 5-[2-isobutoxy-5-(4-methylpiperazine-1-sulfonyl)phenyl]-1-methyl-3-propyl-1,6-dihydropyrazolo[4,3-d]pyrimidine-7-thione

(Step 1) Preparation of 5-(2-isobutoxyphenyl)-1-methyl-3-propyl-1,6-dihydropyrazolo[4,3-d]pyrimidine-7-thione

The title compound was prepared from 5-(2-isobutoxyphenyl)-1-methyl-3-propyl-1,6-dihydropyrazolo[4,3-d]pyrimidin-7-one in accordance with Step 1 of Example 1. (Yield: 81.8%)

¹H-NMR (300 MHz, CDCl₃) δ 12.53 (bs, 1H), 8.44 (dd, J=7.9 Hz, J′=1.7 Hz, 1H), 7.41 (m, 1H), 7.09 (t, J=7.6 Hz, 1H), 7.00 (d, J=8.4 Hz, 1H), 4.50 (s, 3H), 3.96 (d, J=6.5 Hz, 2H), 2.93 (t, J=7.6 Hz, 2H), 2.37 (m, 1H), 1.87 (m, 2H), 1.17 (t, J=6.7 Hz, 6H), 1.03 (t, J=7.4 Hz, 3H)

¹³C-NMR (75 MHz, CDCl₃) δ 172.07, 157.21, 148.30, 146.46, 134.62, 133.08, 132.59, 131.13, 122.08, 119.30, 113.00, 76.66, 39.71, 28.66, 28.06, 22.71, 20.04, 14.49

(Step 2) Preparation of 4-isobutoxy-3-(1-methyl-3-propyl-7-thioxo-6,7-dihydro-1H-pyrazolo[4,3-d]pyrimidin-5-yl)benzenesulfonyl chloride

The title compound was prepared from 5-(2-isobutoxyphenyl)-1-methyl-3-propyl-1,6-dihydropyrazolo[4,3-d]pyrimidine-7-thione in accordance with Step 2 of Example 1. (Yield: 100.0%)

¹H-NMR (300 MHz, CDCl₃) δ 12.17 (bs, 1H), 9.12 (d, J=2.5 Hz, 1H), 8.12 (dd, J=8.9 Hz, J′=2.5 Hz, 1H), 7.27 (d, J=9.0 Hz, 1H), 4.52 (s, 3H), 4.14 (d, J=6.4 Hz, 2H), 2.97 (t, J=7.5 Hz, 2H), 2.46 (r, 1H), 1.86 (m, 2H), 1.21 (d, J=6.7 Hz, 6H), 1.04 (t, J=7.3 Hz, 3H)

¹³C-NMR (75 MHz, CDCl₃) δ 172.27, 161.61, 147.12, 145.70, 137.99, 133.94, 132.76, 131.59, 131.08, 121.10, 114.13, 76.14, 39.84, 28.61, 27.86, 22.66, 19.89, 14.40

(Step 3) Preparation of 5-[2-isobutoxy-5-(4-methylpiperazine-1-sulfonyl)phenyl]-1-methyl-3-propyl-1,6-dihydropyrazolo[4,3-d]pyridine-7-thione

The title compound was prepared from 4-isobutoxy-3-(1-methyl-3-propyl-7-thioxo-6,7-dihydro-1H-pyrazolo[4,3-d]pyrimidin-5-yl)benzenesulfonyl chloride and 1-methylpiperazine in accordance with Step 3 of Example 1. (Yield: 51.2%)

¹H-NMR (300 MHz, CDCl₃) δ12.28 (bs, 1H), 8.84 (d, J=2.4 Hz, 1H), 77.85 (dd, J=8.8 Hz, J′=2.4 Hz, 1H), 7.19 (d, J=8.8 Hz, 1H), 4.53 (s, 3H), 4.08 (d, J=6.5 Hz, 2H), 3.11 (bs, 4H), 2.95 (t, J=7.5 Hz, 2H), 2.51 (m, 4H), 2.43 (m, 1H), 2.28 (s, 3H), 1.86 (m, 2H), 1.20 (t, J=6.7 Hz, 6H), 1.02 (t, J=7.4 Hz, 3H)

¹³C-NMR (75 MHz, CDCl₃) δ 172.31, 160.10, 146.92, 146.51, 134.25, 132.76, 132.39, 131.30, 129.45, 120.45, 113.52; 77.52, 54.40, 46.30, 46.07, 39.81, 28.61, 27.99, 22.60, 19.91, 14.42

### (Example 19)

Preparation of. 5-[2-isobutoxy-5-(4-ethylpiperazine-1-sulfonyl)phenyl]-1-methyl-3-propyl-1,6-dihydropyrazolo[4,3-d]pyrimidine-7-thione

The title compound was prepared from the title compound prepared in Step 2 of Example 18 and 1-ethylpiperazine, in accordance with Step 3 of Example 1. (Yield: 53.0%)

¹H-NMR (300 MHz, CDCl₃) δ 12.28 (bs, 1H), 8.84 (d, J=2.4 Hz, 1H), 7.86 (dd, J=8.8 Hz, J′=2.4 Hz, 1H), 7.18 (d, J=8.8 Hz, 1H), 4.53 (s, 31), 4.08 (d, J=6.5 Hz, 2H), 3.11 (bs, 4H), 2.95 (t, J=7.5 Hz, 2H), 2.55 (m, 4H), 2.47-2.38 (m, 3H), 1.86 (m, 2H), 1.20 (t, J=6.7 Hz, 6H), 1.06-0.99 (n, 6H)

¹³C-NMR (75 MHz, CDCl₃) δ 172.32, 160.10, 146.92, 146.51, 134.25, 132.76, 132.45, 131.37, 129.31, 120.44, 113.50, 77.53, 52.31, 52.19, 46.46, 39.81, 28.61, 27.99, 22.29, 19.91, 14.41, 12.30

### (Example 20)

Preparation of 5-{5-[4-(2-hydroxyethyl)piperazine-1-sulfonyl]-2-isobutoxy-phenyl}-1-methyl-3-propyl-1,6-dihydropyrazolo[4,3-d]pyrimidine-7-thione

The title compound was prepared from the title compound prepared in Step 2 of Example 18 and 1-(2-hydroxyethyl)piperazine, in accordance with Step 3 of Example 1. (Yield: 56.9%)

¹H-NMR (300 MHz, CDCl₃) δ 12.28 (bs, 1H), 8.82 (d, J=2.4 Hz, 1H), 7.85(dd, J=8.8 Hz, r=2.4 Hz, 1H), 7.21 (d, J=8.8 Hz, 111), 4.53 (s, 3H), 4.10 (d, J=6.4 Hz, 2H), 3.57 (t, J=5.3 Hz, 2H), 3.10 (bs, 4H), 2.95 (t, J=7.5 Hz, 2H), 2.62 (m, 4H), 2.55 (t, J=5.3 Hz, 2H), 2.43 (m, 1H), 2.30 (bs, 1H), 1.86 (m, 2H), 1.20 (d, J=6.7 Hz, 6H), 1.02 (t, J=7.4 Hz, 3H)

¹³C-NMR (75 MHz, CDCl₃) δ 172.29, 160.16, 146.88, 146.53, 134.20, 132.74, 132.39, 131.26, 129.20, 120.53, 113.59, 59.31, 58.14, 52.34, 46.52, 39.82, 28.63, 27.97, 22.59, 19.93, 14.43

### (Example 21)

Preparation of 5-[2-isobutoxy-5-(morpholine-4-sulfonyl)phenyl]-1-methyl-3-propyl-1,6-dihydropyrazolo[4,3-d]pyrimidine-7-thione

The title compound was prepared from the title compound prepared in Step 2 of Example 18 and morpholine, in accordance with Step 3 of Example 1. (Yield: 53.3%)

¹H-NMR (300 MHz, CDCl₃) δ 12.27 (bs, 1H), 8.83 (d, J=2.4 Hz, 1H), 7.86 (dd, J=8.8 Hz, J′=2.4 Hz, 1H), 7.22 (d, J=8.8 Hz, 1H), 4.53 (s, 3), 4.10 (d, J=6.4 Hz, 2H), 3.77 (t, J=4.7 Hz, 4H) 3.06 (t, J=4.6 Hz, 4H), 2.95 (t, J=7.5 Hz, 2H), 2.43 (m, 1H), 1.86 (m, 2H), 1.21 (d, J=6.7 Hz, 6H), 1.02 (t, J=7.4 Hz, 3H)

¹³C-NMR (75 MHz, CDCl₃) δ 172.31, 160.23, 146.90, 146.47, 134.20, 132.76, 132.41, 131.31, 129.11, 120.60, 113.63, 77.53, 66.49, 46.40, 39.82, 28.64, 27.98, 22.60, 19.93, 14.42

### (Example 22)

Preparation of 1-methyl-5-[2-(3-methylbutoxy)-5-(4-methylpiperazine-1-sulfonyl)phenyl]-3-propyl-1,6-dihydropyrazolo[4,3-d]pyrimidine-7-thione

(Step 1) Preparation of 1-methyl-5-[2-(3-methylbutoxy)phenyl]-3-propyl-1,6-dihydropyrazolo[4,3-d]pyrimidine-7-thione

The title compound was prepared from 1-methyl-5-[2-(3-methylbutoxy)phenyl]-3-propyl-1,6-dihydropyrazolo[4,3-d]pyrimidin-7-one in accordance with Step 1 of Example 1. (Yield: 88.0%)

¹H-NMR (300 MHz, CDCl₃) δ 12.47 (bs, 1H), 8.45 (d, J=7.9 Hz, 1H), 7.42 (m, 1H), 7.10 (t, J=7.6 Hz, 1H), 7.03 (d, J=8.4 Hz, 1H), 4.50 (s, 3H), 4.23 (t, J=6.1 Hz, 2H), 2.93 (t, J=7.6 Hz, 2H), 2.02-1.83 (r, 5H), 1.03 (t, J=7.3 Hz, 3H), 1.02 (t, J=6.3 Hz, 6H)

¹³C-NMR (75 MHz, CDCl₃) δ 172.06, 157.17, 148.27, 146.47, 134.66, 133.08, 132.60, 131.07, 122.10, 119.19, 112.95, 68.44, 39.37, 38.06, 28.06, 25.47, 22.84, 22.70, 14.48

(Step 2) Preparation of 4-(3-methylbutoxy)-3-(1-methyl-3-propyl-7-thioxo-6,7-dihydro-1H-pyrazolo[4,3-d]pyrimidin-5-yl)benzenesulfonyl chloride

The title compound was prepared from 1-methyl-5-[2-(3-methylbutoxy)phenyl]-3-propyl-1,6-dihydropyrazolo[4,3-d]pyrimidine-7-thione in accordance with Step 1 of Example 1. (Yield: 97.5%)

¹H-NMR (300 MHz, CDCl₃) δ 12.25 (bs, 1H), 9.12 (d, J=2.6 Hz, 1H), 8.12 (dd, J=8.9 Hz, J′=2.6 Hz, 1H), 7.30 (d, J=8.0 Hz, 1H), 4.51 (s, 3H), 4.42 (t, J=6.0 Hz, 2H), 2.97 (t, J=7.5 Hz, 2H), 2.06-1.99 (m, 31H), 1.87 (1,2H), 1.06-1.01 (m, 9H)

¹³C-NMR (75 MHz, CDCl₃) δ 172.18, 161.56, 147.07, 145.66, 137.89, 133.94, 132.73, 131.59, 130.93, 120.88, 114.13, 70.04, 39.80, 37.75, 27.86, 25.43, 22.75, 22.64, 14.41

(Step 3) Preparation of 1-methyl-5-[2-(3-methylbutoxy)-5-(4-methylpiperazine-1-sulfonyl)phenyl]-3-propyl-1,6-dihydropyrazolo[4,3-d]pyrimidine-7-thione

The title compound was prepared from 4-(3-methylbutoxy)-3-(1-methyl-3-propyl-7-thioxo-6,7-dihydro-1H-pyrazolo[4,3-d]pyrimidin-5-yl)benzenesulfonyl chloride and 1-methylpiperazine in accordance with Step 1 of Example 1. (Yield: 75.3%)

¹H-NMR (300 MHz, CDCl₃) δ 12.36 (bs, 1H), 8.84 (d, J=2.4 Hz, 1H), 7.85 (dd, J=8.8 Hz, J′=2.4 Hz, 1H), 7.21 (d, J=8.8 Hz, 1H), 4.53 (s, 3H), 4.35 (t, J=6.1 Hz, 2), 3.10 (bs, 4H)12.95 (t, J=7.5 Hz, 2H), 2.50 (t, J=4.7 Hz, 4H), 2.28 (s, 3H), 2.05-1.94 (m, 3H), 1.86 (m, 2H), 1.05-0.99 (m, 9H)

¹³C-NMR (75 MHz, CDCl₃) δ 172.22, 160.02, 146.87, 146.47, 134.24, 132.73, 132.39, 131.17, 129.34, 120.24, 113.48, 69.43, 54.40, 46.32, 46.09, 39.76, 37.82, 27.98, 25.43, 22.76, 22.57, 14.41

### (Example 23)

Preparation of 1-methyl-5-[2-(3-methylbutoxy)-5-(4-ethylpiperazine-1-sulfonyl)phenyl]-3-propyl-1,6-dihydropyrazolo[4,3-d]pyrimidine-7-thione

The title compound was prepared from the title compound prepared in Step 2 of Example 22 and ethylpiperazine, in accordance with Step 3 of Example 1. (Yield: 80.4%)

¹H-NMR (300 MHz, CDCl₃) δ 12.36 (bs, 1H), 8.85 (d, J=2.4 Hz, 1H), 7.85 (dd, J=8.8 Hz, J′=2.4 Hz, 1H), 7.21 (d, J=8.8 Hz, 1H), 4.53 (s, 31, 4.35 (t, J=6.1 Hz, 21), 3.11 (bs, 41) 2.95 (t, J=7.5 Hz, 2H), 2.55 (t, J=4.7 Hz, 4H), 2.41 (q, J=7.2 Hz, 2H), 2.05-1.98 (m, 3H), 1.86 (m, 2H), 1.05-0.99 (m, 12H)

¹³C-NMR (75 MHz, CDCl₃) δ 172.23, 160.02, 146.88, 146.47, 134.25, 132.73, 132.46, 131.24, 129.17, 120.23, 113.47, 69.43, 52.29, 52.18, 46.47, 39.76, 37.82, 27.98, 25.43, 22.76, 22.57, 14.42, 12.33

### (Example 24)

Preparation of 5-[5-[4-(2-hydroxyethyl)piperazine-1-sulfonyl]-2-(3-methylbutoxy)phenyl]-1-methyl-3-propyl-1,6-dihydropyrazolo[4,3-d]pyrimidine-7-thione

The title compound was prepared from the title compound prepared in Step 2 of Example 22 and 1-(2-hydroxyethyl)piperazine, in accordance with Step 3 of Example 1. (Yield: 85.8%)

¹H-NMR (300 MHz, CDCl₃) δ 12.36 (bs, 1H), 8.83 (d, J=2.4 Hz, 1H), 7.85(dd, J=8.8 Hz, r=2.4 Hz, 1H), 7.23 (d, J=8.8 Hz, 1H), 4.53 (s, 3H), 4.36 (t, J=6.1 Hz, 2H), 3.58 (t, J=5.3 Hz, 2H), 3.10 (bs, 4H), 2.95 (t, J=7.5 Hz, 2H), 2.62 (m, 4H), 2.55 (t, J=5.3 Hz, 2H), 2.36 (bs, 1H), 2.08-1.94(m, 3H), 1.86 (r, 2H), 1.05-1.00 (m, 9H)

¹³C-NMR (75 MHz, CDCl₃) δ 172.23, 160.10, 146.87, 146.49, 134.22, 132.73, 132.39, 131.17, 129.18, 120.35, 113.54, 69.43, 59.33, 58.16, 52.3, 46.51, 39.78, 37.84, 27.97, 25.43, 22.78, 22.58, 14.43

### (Example 25)

Preparation of 1-methyl-5-[2-(3-methylbutoxy)-5-(morpholine-4-sulfonyl)phenyl]-3-propyl-1,6-dihydropyrazolo[4,3-d]pyrimidine-7-thione

The title compound was prepared from the title compound prepared in Step 2 of Example 22 and morpholine, in accordance with Step 3 of Example 1. (Yield: 86.0%)

¹H-NMR (300 MHz, CDCl₃) δ 12.35 (bs, 1H), 8.84 (d, J=2.4 Hz, 1H), 7.85 (dd, J=8.8 Hz, J′=2.4 Hz, 1H), 7.24 (d, J=8.8 Hz, 1H), 4.53 (s, 3H), 4.37 (t, J=6.1 Hz, 2H), 3.77 (t, J=4.6 Hz, 4H), 3.06 (t, J=4.6 Hz, 4H) 2.95 (t, J=7.5 Hz, 2H), 2.06-1.95 (r, 3H), 1.85 (r, 2H), 1.05 (d, J=6.2 Hz, 6H), 1.02 (t, J=7.2 Hz, 3H)

¹³C-NMR (75 MHz, CDCl₃) δ 172.24, 160.17, 146.88, 146.43, 134.21, 132.74, 132.42, 131.20, 129.06, 120.41, 113.59, 69.46, 66.48, 46.39, 39.79, 37.84, 27.98, 25.44, 22.78, 22.58, 14.41

### [Experimental Example 1]

### Inhibitory Activities Against Phosphodiesterase (PDE) I, III, V and VI

In order to identify the effects of compounds of formula 1 according to the present invention on erectile dysfunction, inhibitory activities against phosphodiesterase V and other isozymes (I, III and VI) were evaluated in accordance with the following procedure.

Phosphodiesterases I and III are associated with the side effects affecting the cardiovascular system, and phosphodiesterase VI is associated with the side effects affecting the eyes. Accordingly, excellent drugs for treating erectile dysfunction must satisfy the following requirements: i) high inhibitory activity against phosphodiesterase V, and ii) low inhibitory activity against phosphodiesterases I, III and VI.

Phosphodiesterases I, III and V were isolated from diaphragm kidney cortex of rat, phosphodiesterase VI was isolated from retina of rat. Inhibitory activities against the isolated enzymes were evaluated by the method of Thompson and Appleman (Thompson and Appleman, Biochemistry, 1971, 10, 311-316). A reaction mixture for enzyme activity [Total volume 100 μl: PDE enzymes (Column fraction 20˜40 μl), 10 nM CaCl₂ and 20 μl M calmodulin (10 μl addition for PDE I), [³H]-cAMP, [³H]-cGMP(1 μCi/μl), compound of formula 1 (0.01 nM˜1 μM), 50 mM Tris-HCl buffer solution (pH 7.4), 15 mM MgCl₂, distilled water] was incubated in a water bath at 30° C. for 30 minutes, and then heat-denatured at 100° C. for 2 minutes to denature enzymes. After the reaction mixture was cooled down on ice, 250 μg/ml venom was added thereto. The resulting mixture was incubated at 30° C. for 10 minutes, and then 0.5 ml of cold d-H₂O was added thereto to obtain a specimen. Guanine was separated from the specimen using an anion exchange resin (DEAE Sephacel A-25 anion exchange column). To measure the IC₅₀ of specimen, radioactivity was counted by β-counter after treatment with 10 ml of scintillation cocktail solution.

TABLE 1

IC₅₀(nM)

| Compound No | PDE I | PDE III | PDE V | PDE VI |
|---|---|---|---|---|
| 5 | 1890 | 1250 | 0.59 | 60 |
| 7 | 110 | 3020 | 0.40 | 56 |
| 9 | 2430 | 5630 | 0.62 | 327 |
| 10 | 620 | 1030 | 0.58 | 83 |
| 12 | 10 | 1080 | 0.46 | 35 |
| Sildenafil | 2250 | 1750 | 6.86 | 99 |

As can be seen from Table 1, the compounds of formula 1 (pyrazolopyrimidinethione compounds) according to the present invention exhibit higher inhibitory activities against phosphodiesterase V as well as lower inhibitory activities against phosphodiesterase isozymes (I, III and VI), compared to sildenafil. Accordingly, the compounds of formula 1 according to the present invention were proved to be useful drugs for treating erectile dysfunction.

### [Experimental Example 2]

### Acute Toxicity when Administered Orally to Mouse

In order to evaluate the toxicity of the compounds of formula 1 according to the present invention, an experiment was performed as described below.

The compound prepared in the Example 5 was administered orally to four ICR mice (2 male, 2 female) once at a dose of 128, 320, 800, 2000 and 5000 mg/kg, respectively. 7 days after the administration, mortality rate, general symptoms, changes in weight, and autopsy results were recorded. As a result, no negative effects were observed even at a dose of 5000 mg/kg.

In conclusion, the compound prepared in the Example 5 has no toxicity when administered orally at a dose of not more than 5000 mg/kg. Also, because the minimal lethal doses are more than 5000 mg/kg in both male and female rats, the compounds according to the present invention were demonstrated to be safe

### INDUSTRIAL APPLICABILITY

As can be seen from the foregoing, the present invention provides pyrazolopyrimidinethione compounds having excellent curative effects on erectile dysfunction and few side effects, and pharmaceutical compositions comprising the compounds for treating erectile dysfunction.

While this invention has been described in connection with what is presently considered to be the most practical and preferred embodiment, it is to be understood that the invention is not limited to the disclosed embodiment, but, on the contrary, it is intended to cover various modifications and variations within the spirit and scope of the appended claims.

1. A compound of formula 1:
<Formula 1> 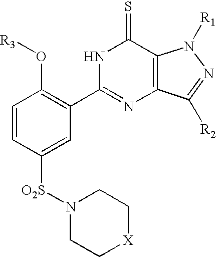
wherein
R₁ and R₂ are independently each hydrogen atom, a C₁-C₆ alkyl group, or a C₃-C₆ cycloalkyl group,
R₃ is a C₁-C₆ alkyl group, C₃-C₆ cycloalkyl group or C₃-C₆ alkenyl group which is substituted or unsubstituted,
X is O or NR₄, and
R₄ is hydrogen atom, or a C₁-C₆ alkyl group, a C₃-C₆ cycloalkyl group or a C₃-C₆ alkenyl group which is unsubstituted or substituted with OH or an alkoxy group, or pharmacologically acceptable salts or hydrates thereof.

2. The compound as set forth in claim 1, wherein the compound is selected from the group consisting of:
1) 5-[2-methoxy-5-(4-methylpiperazine-1-sulfonyl)phenyl]1-methyl-3-propyl-1,6-dihydropyrazolo[4,3-d]pyridine-7-thione,
2) 5-[2-methoxy-5-(4-ethylpiperazine-1-sulfonyl)phenyl]-1-methyl-3-propyl-1,6-dihydropyrazolo[4,3-d]pyrimidine-7-thione,
3) 5-{5-[4-(2-hydroxyethyl)piperazine-1-sulfonyl]-2-methoxyphenyl)-1-methyl-3-propyl-1,6-dihydropyrazolo[4,3-d]pyrimidine-7-thione,
4) 5-[2-methoxy-5-(morpholine-4-sulfonyl)phenyl]-1-methyl-3-propyl-1,6-dihydropyrazolo[4,3-d]pyrimidine-7-thione,
5) 5-[2-ethoxy-5-(4-methylpiperazine-1-sulfonyl)phenyl]-1-methyl-3-propyl-1,6-dihydropyrazolo[4,3-d]pyrimidine-7-thione,
6) 5-[2-ethoxy-5-(4-ethylpiperazine-1-sulfonyl)phenyl]-1-methyl-3-propyl-1,6-dihydropyrazolo[4,3-d]pyrimidine-7-thione,
7) 5-(2-ethoxy-5-[4-(2-hydroxyethyl)piperazine-1-sulfonyl]phenyl}-1-methyl-3-propyl-1,6-dihydropyrazolo[4,3-d]pyrimidine-7-thione,
8) 5-[2-ethoxy-5-(morpholine-4-sulfonyl)phenyl]-1-methyl-3-propyl-1,6-dihydropyrazolo[4,3-d]pyrimidine-7-thione,
9) 5-[2-ethoxy-5-(piperazine-1-sulfonyl)phenyl]-1-methyl-3-propyl-1,6-dihydropyrazolo[4,3-d]pyrimidine-7-thione,
10) 1-methyl-5-[5-(4-methylpiperazine-1-sulfonyl)-2-propoxyphenyl]-3-propyl-1,6-dihydropyrazolo[4,3-d]pyrimidine-7-thione,
11) 5-[5-(4-ethylpiperazine-1-sulfonyl)-2-propoxyphenyl]-1-methyl-3-propyl-1,6-dihydropyrazolo[4,3-d]pyrimidine-7-thione,
12) 5-{5-[4-(2-hydroxyethyl)piperazine-1-sulfonyl]-2-propoxyphenyl}-1-methyl-3-propyl-1,6-dihydropyrazolo[4,3-d]pyrimidine-7-thione,
13) 1-methyl-5-[5-(morpholine-4-sulfonyl)-2-propoxyphenyl]-3-propyl-1,6-dihydropyrazolo[4,3-d]pyrimidine-7-thione,
14) 5-[2-butoxy-5-(4-methylpiperazine-1-sulfonyl)phenyl]-1-methyl-3-propyl-1,6-dihydropyrazolo[4,3-d]pyrimidine-7-thione,
15) 5-[2-butoxy-5-(4-ethylpiperazine-1-sulfonyl)phenyl]-1-methyl-3-propyl-1,6-dihydropyrazolo[4,3-d]pyrimidine-7-thione,
16) 5-(2-butoxy-5-[4-(2-hydroxyethyl)piperazine-1-sulfonyl]phenyl)-1-methyl-3-propyl-1,6-dihydropyrazolo[4,3-d]pyrimidine-7-thione,
17) 5-[2-butoxy-5-(morpholine-4-sulfonyl)phenyl]-1-methyl-3-propyl-1,6-dihydropyrazolo[4,3-d]pyrimidine-7-thione,
18) 5-[2-isobutoxy-5-(4-methylpiperazine-1-sulfonyl)phenyl]-1-methyl-3-propyl-1,6-dihydropyrazolo[4,3-d]pyrimidine-7-thione,
19) 5-[2-isobutoxy-5-(4-ethylpiperazine-1-sulfonyl)phenyl]-1-methyl-3-propyl-1,6-dihydropyrazolo[4,3-d]pyrimidine-7-thione,
20) 5-{5-[4-(2-hydroxyethyl)piperazine-1-sulfonyl]-2-isobutoxyphenyl}-1-methyl-3-propyl-1,6-dihydropyrazolo[4,3-d]pyrimidine-7-thione,
21) 5-[2-isobutoxy-5-(morpholine-4-sulfonyl)phenyl]-1-methyl-3-propyl-1,6-dihydropyrazolo[4,3-d]pyrimidine-7-thione,
22) 1-methyl-5-[2-(3-methylbutoxy)-5-(4-methylpiperazine-1-sulfonyl)phenyl]-3-propyl-1,6-dihydropyrazolo[4,3-d]pyrimidine-7-thione,
23) 1-methyl-5-[2-(3-methylbutoxy)-5-(4-ethylpiperazine-1-sulfonyl)phenyl]-3-propyl-1,6-dihydropyrazolo[4,3-d]pyrimidine-7-thione,
24) 5-[5-[4-(2-hydroxyethyl)piperazine-1-sulfonyl]-2-(3-methylbutoxy)phenyl]-1-methyl-3-propyl-1,6-dihydropyrazolo[4,3-d]pyrimidine-7-thione, and
25) 1-methyl-5-[2-(3-methylbutoxy)-5-(morpholine-4-sulfonyl)phenyl]-3-propyl-1,6-dihydropyrazolo[4,3-d]pyrimidine-7-thione.

3. A method for preparing a pyrazolopyrimidinethione compound, comprising the steps of:
converting a pyrazolopyrimidinone compound of formula 2 into a pyrazolopyrimidinethione compound of formula 3 via thionation;
converting the pyrazolopyrimidinethione compound of formula 3 into a chlorosulfonated compound of formula 4 via chlorosulfonation; and
reacting the chlorosulfonated compound of formula 4 with an amine in a solvent: 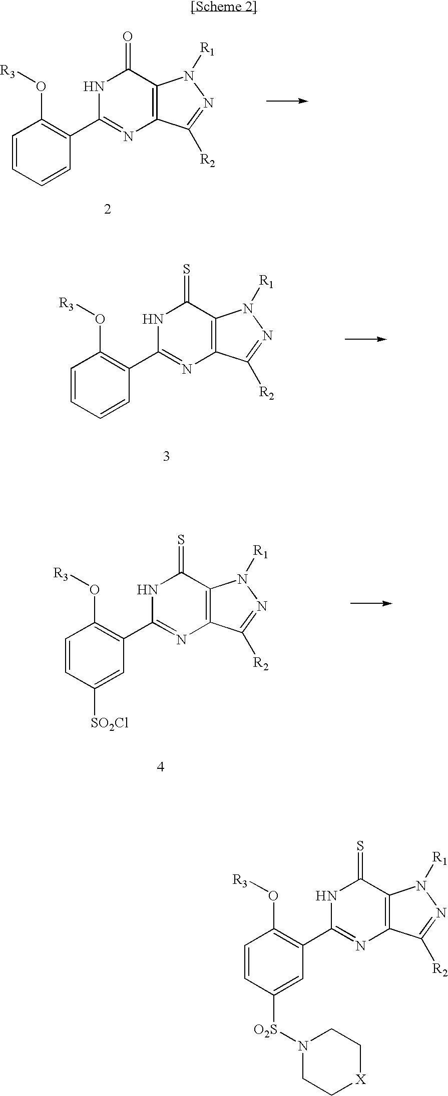
wherein
R₁, R₂, R₃ and X are as defined in claim 1.

4. The method as set forth in claim 3, wherein the thionation is carried out by reacting phosphorus pentasulfide, 2,4-bis(4-methoxyphenyl)-1,3-dithia-2,4-diphosphetane-2,4-disulfide or derivatives thereof in tetrahydrofuran, dioxane, pyridine, benzene, xylene or toluene at room temperature or refluxing temperature.

5. The method as set forth in claim 3, wherein the chlorosulfonation is carried out by stirring the pyrazolopyrimidinethione compound of formula 3, 5 to 20 equivalents of chlorosulfonic acid and 2 to 10 equivalents of thionyl chloride at 0° C. or at room temperature.

6. The method as set forth in claim 3, wherein the chlorosulfonated compound of formula 4 is reacted with 2 to 5 equivalents of secondary amine, or a mixture of 1 equivalent of secondary amine and 1 to 5 equivalents of tertiary amine, in alkanol, tetrahydrofuran, water, acetonitrile, pyridine, dimethylformamide or N,N-dimethylacetamide.

7. The method as set forth in claim 6, wherein the secondary amine is piperazine, morpholine or piperazine derivatives, and the tertiary amine is triethylamine or pyridine.

8. A method for preparing a pyrazolopyrimidinethione compound by thionating a pyrazolopyrimidinone compound of formula 5:
[Scheme 3] 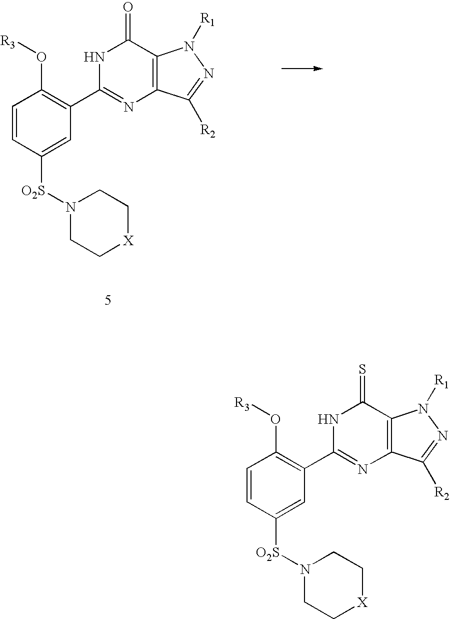
wherein
R₁, R₂, R₃ and X are as defined in claim 1.

9. The method as set forth in claim 8, wherein the thionation is carried out by reacting phosphorus pentasulfide, 2,4-bis(4-methoxyphenyl)-1,3-dithia-2,4-diphosphetane-2,4-disulfide or derivatives thereof, in tetrahydrofuran, dioxane, pyridine or toluene at room temperature or refluxing temperature.

10. A pharmaceutical composition for treating erectile dysfunction comprising the pyrazolopyrimidinethione compound, pharmacologically acceptable salts or hydrates thereof as set forth in claim 1.

11. The pharmaceutical composition as set forth in claim 10, wherein the pharmaceutical composition is formulated into oral administration forms.

12. The pharmaceutical composition as set forth in claim 10, wherein the pharmaceutical composition is formulated into injection solutions.
